SEQUENCE LISTING (27 sequences)

SEQ ID NO: 1 - DNA, 492 bp, Homo sapiens
gctagcgctagcgccaccatgctggagaagctcatgggcgctgactccctgcaattgttcaggtcccgctacactctgggcaagatctatttcatcggcttccagaagtccattctgctgtccaaaagtgagaatagcctcaacagcatcgctaaggagaccgaggagggaagagaaaccgtgacccgcaaggaggggtggaagaggcgtcacgaagacgggtatctcgagatggcccagaggcacctccagcggagcctgtgtccctgggtctcctacctcccccagccttacgccgagctcgaggaggtgtcgtctcacgtcggcaaggttttcatggcgcggaactacgagtttctggcttacgaggcctcgaaggaccgccgccagcccctggagcgcatgtggacctgcaactacaaccagcagaaggatcagtcctgtaaccacaaggagatcaccagtaccaaagcggagtgaggatccggatcc

SEQ ID NO: 2 - DNA, 20 bp, Artificial Sequence
  Overlapping oligonucleotide HDAOAS1
tccggctagcgctagcgcca

SEQ ID NO: 3 - DNA, 40 bp, Artificial Sequence
  Overlapping oligonucleotide HDAOAS2
ccatgctggagaagctcatgggcgctgactccctgcaatt

SEQ ID NO: 4 - DNA, 40 bp, Artificial Sequence
  Overlapping oligonucleotide HDAOAS3
gttcaggtcccgctacactctgggcaagatctatttcatc

SEQ ID NO: 5 - DNA, 40 bp, Artificial Sequence
  Overlapping oligonucleotide HDAOAS4
ggcttccagaagtccattctgctgtccaaaagtgagaata

SEQ ID NO: 6 - DNA, 40 bp, Artificial Sequence
  Overlapping oligonucleotide HDAOAS5
gcctcaacagcatcgctaaggagaccgaggagggaagaga

SEQ ID NO: 7 - DNA, 40 bp, Artificial Sequence
  Overlapping oligonucleotide HDAOAS6
aaccgtgacccgcaaggaggggtggaagaggcgtcacgaa

SEQ ID NO: 8 - DNA, 40 bp, Artificial Sequence
  Overlapping oligonucleotide HDAOAS7
gacgggtatctcgagatggcccagaggcacctccagcgga

SEQ ID NO: 9 - DNA, 40 bp, Artificial Sequence
  Overlapping oligonucleotide HDAOAS8
gcctgtgtccctgggtctcctacctcccccagccttacgc

SEQ ID NO: 10 - DNA, 40 bp, Artificial Sequence
  Overlapping oligonucleotide HDAOAS9
cgagctcgaggaggtgtcgtctcacgtcggcaaggttttc

SEQ ID NO: 11 - DNA, 40 bp, Artificial Sequence
  Overlapping oligonucleotide HDAOAS10
atggcgcggaactacgagtttctggcttacgaggcctcga

SEQ ID NO: 12 - DNA, 40 bp, Artificial Sequence
  Overlapping oligonucleotide HDAOAS11
aggaccgccgccagcccctggagcgcatgtggacctgcaa

SEQ ID NO: 13 - DNA, 40 bp, Artificial Sequence
  Overlapping oligonucleotide HDAOAS12
ctacaaccagcagaaggatcagtcctgtaaccacaaggag

SEQ ID NO: 14 - DNA, 40 bp, Artificial Sequence
  Overlapping oligonucleotide HDAOAS13
atcaccagtaccaaagcggagtgaggatccggatcccgtt

SEQ ID NO: 15 - DNA, 20 bp, Artificial Sequence
  Overlapping oligonucleotide HDAOAAS1
aacgggatccggatcctcac

SEQ ID NO: 16 - DNA, 40 bp, Artificial Sequence
  Overlapping oligonucleotide HDAOAAS2
tccgctttggtactggtgatctccttgtggttacaggact

SEQ ID NO: 17 - DNA, 40 bp, Artificial Sequence
  Overlapping oligonucleotide HDAOAAS3
gatccttctgctggttgtagttgcaggtccacatgcgctc

SEQ ID NO: 18 - DNA, 40 bp, Artificial Sequence
  Overlapping oligonucleotide HDAOAAS4
caggggctggcggcggtccttcgaggcctcgtaagccaga

SEQ ID NO: 19 - DNA, 40 bp, Artificial Sequence
  Overlapping oligonucleotide HDAOAAS5
aactcgtagttccgcgccatgaaaaccttgccgacgtgag

SEQ ID NO: 20 - DNA, 40 bp, Artificial Sequence
  Overlapping oligonucleotide HDAOAAS6
acgacacctcctcgagctcggcgtaaggctgggggaggta

SEQ ID NO: 21 - DNA, 40 bp, Artificial Sequence
  Overlapping oligonucleotide HDAOAAS7
ggagacccagggacacaggctccgctggaggtgcctctgg

SEQ ID NO: 22 - DNA, 40 bp, Artificial Sequence
  Overlapping oligonucleotide HDAOAAS8
gccatctcgagatacccgtcttcgtgacgcctcttccacc

SEQ ID NO: 23 - DNA, 40 bp, Artificial Sequence
  Overlapping oligonucleotide HDAOAAS9
cctccttgcgggtcacggtttctcttccctcctcggtctc

SEQ ID NO: 24 - DNA, 40 bp, Artificial Sequence
  Overlapping oligonucleotide HDAOAAS10
cttagcgatgctgttgaggctattctcacttttggacagc

SEQ ID NO: 25 - DNA, 40 bp, Artificial Sequence
  Overlapping oligonucleotide HDAOAAS11
agaatggacttctggaagccgatgaaatagatcttgccca

SEQ ID NO: 26 - DNA, 40 bp, Artificial Sequence
  Overlapping oligonucleotide HDAOAAS12
gagtgtagcgggacctgaacaattgcagggagtcagcgcc

SEQ ID NO: 27 - DNA, 40 bp, Artificial Sequence
  Overlapping oligonucleotide HDAOAAS13
catgagcttctccagcatggtggcgctagcgctagccgga

(19) United States
(12) Patent Application Publication
(10) Pub. No.: US 2008/0182296 A1
(43) Pub. Date: Jul. 31, 2008

(54) PCR-DIRECTED GENE SYNTHESIS FROM LARGE NUMBER OF OVERLAPPING OLIGODEOXYRIBONUCLEOTIDES

(76) Inventors: Pranab K. Chanda, West Windsor, NJ (US); Bart W. Nieuwenhuijsen, Philadelphia, PA (US)

Correspondence Address: FITZPATRICK CELLA (WYETH), 30 ROCKEFELLER PLAZA, NEW YORK, NY, 10112-3800, US

(21) Appl. No.: 12/023,756

(22) Filed: Jan. 31, 2008

Related U.S. Application Data
(60) Provisional application No. 60/898,448, filed on Jan. 31, 2007.

(51) Int. Cl. C12P 21/04; C12P 21/00; C07K 16/00; C07H 21/04

(52) U.S. Cl. 435/69.1; 435/91.2; 536/23.2; 530/350

(57) ABSTRACT

The present invention provides methods of PCR-directed gene synthesis that may be used for all genes, including those with a high G+C content and/or a long sequence. The invention relates to methods of gene synthesis using overlapping oligonucleotides and polymerase chain reaction (PCR), wherein several PCR parameters, e.g., the concentration of overlapping oligonucleotides, the type of DNA polymerase used, and the number of PCR amplification cycles, are optimized. Additionally, the invention relates to oligonucleotide design that allows for increased protein expression of synthesized genes.

### RELATED APPLICATIONS

This application claims the benefit of priority from U.S. Provisional Patent Application No. 60/898,448, filed Jan. 31, 2007, which is hereby incorporated by reference herein in its entirety.

### FIELD

The invention relates to methods of gene synthesis using overlapping oligonucleotides and polymerase chain reactions (PCRs), wherein several PCR parameters, e.g., the concentration of oligonucleotides, the type of DNA polymerase used, the number of PCR amplification cycles, etc., are optimized. The present invention is useful for synthesis of all genes, including those with a high G+C content and/or a long sequence. Additionally, the invention relates to oligonucleotide design that allows for increased protein expression of synthesized genes.

### BACKGROUND

In some research applications, e.g., in biochemical and structural studies using various host expression systems, it is desirable to obtain high levels of gene expression. Synthetic DNA, e.g., a synthetic gene, is a powerful molecular tool in many research applications because it allows the manipulation of gene sequences (e.g., by codon optimization) to obtain, e.g., high levels of gene expression, constructs of mosaic fusion proteins, constructs of linear recombinant DNA, e.g., expression vectors, targeting constructs for gene knockout technology, etc.

DNA can be synthesized chemically through traditional means. However, during the past three decades, several other gene synthesis methods have been described, e.g., the synthesis of DNA from oligonucleotides by a ligation method (Smith et al. (1982) Nucleic Acids Res. 10:4467-82; Edge et al. (1983) Nucleic Acids Res. 11:6419-35; Jay et al. (1984) J. Biol. Chem. 259:6311-17; Sproat et al. (1985) Nucleic Acids Res. 13:2959-77; Ecker et al. (1987) J. Biol. Chem. 262:3524-27; Ashman et al. (1989) Protein Eng. 2:387-91; Heyneker et al. (1976) Nature 748-52; Itakura et al. (1977) Science 198:1056-63; Goeddel et al. (1979) Proc. Natl. Acad. Sci. USA 76:106-10), the FokI method (Mandecki et al. (1988) Gene 68:101-07), a self-priming PCR method (Dillon and Rosen (1990) Biotechniques 9:298-300, Prodromou et al. (1992) Protein Eng. 5:827-29; Cicarelli, et al. (1991) Nucleic Acids Res. 19:6007-13; Hayashi et al. (1994) Biotechniques 17:310-15), and a template directed ligation method (Srizhov et al. (1996) Proc. Natl. Acad. Sci. USA 93:15012-17). Such methods are preferable to chemical gene synthesis because they are simple, rapid, and cost effective.

More recently, methods for synthesizing DNA with long sequences have been reported (Xinxin et al. (2003) Nucleic Acids Res. 31(22):e13; Shevchuk et al. (2004) Nucleic Acids Res. 32:e19; Gao et al. (2004) Biotechnol. Prog. 20:443-48). Due to its simplicity and speed, a particularly appealing method of gene synthesis involves assembly by polymerase chain reaction (PCR) from overlapping oligodeoxyribonucleotides (Stemmer et al. (1995) Gene 164:49-53; Hoover and Lubkowski (2002) Nucleic Acids Res. 30:e43; Gao et al. (2004) supra). These overlapping oligodeoxyribonucleotides are designed to code for the entire sense (+) and antisense (−) strands of DNA. The overlapping oligodeoxyribonucleotides are assembled using “assembly PCR” to generate a template DNA for the gene of interest. Following assembly PCR, the template DNA of the gene of interest is amplified by the two separate and distinct outermost overlapping oligonucleotides, each of which is, respectively, complementary to the sense and antisense strands of the template DNA. The resulting amplified DNA may then be cloned into a vector suitable for a variety of applications (Gao et al. (2004) supra).

Often, overlapping oligonucleotides are designed to allow for optimal expression of the synthesized gene. For example, the codon optimization program, UpGene, breaks a given DNA sequence into triplets and replaces some codons with codons coding for, e.g., equivalent amino acids (based on degeneracy of the genetic code); these replacement codons are more frequently used by a given organism and will increase expression of the protein (Gao et al. (2004) supra). The optimized sequence, including all necessary overlapping oligonucleotides for gene synthesis, is displayed in the output window. The availability of free Web-based DNA codon-optimization computer software (e.g., Hoover and Lubkowski (2002) supra; Gao et al. (2004) supra; Grote et al. (2005) Nucleic Acids Res. 33:W526-31; Withers-Martinez et al. (1999) Protein Eng. 12:1113-20; Richardson et al. (2006) Genome Res. 16:550-56; Jayraj et al. (2005) Nucleic Acids Res. 33:3011-16; Raghava and Sahni (1994) Biotechniques 16:1116-23; DNA Builder (Pacific Northwest National Laboratory, WA)) automates and simplifies the overlapping oligonucleotide design process.

However, these methods of PCR-directed gene synthesis (e.g., Gao et al. (2004) supra; Hoover and Lubkowski (2002) supra) have proven to be inapplicable in some situations, and often fail where the gene of interest has, e.g., a high G+C content. In addition, previously published PCR-directed gene synthesis methods (Stemmer et al. (1995) supra; Gao et al. (2004) supra; Hoover and Lubkowski (2002) supra) require up to 55 cycles of assembly PCR and up to 25 cycles of amplification PCR, and/or utilize DNA polymerases such as Taq or Pfu, which increases the potential for synthesizing genes with numerous mutations.

### SUMMARY

The present invention provides methods of PCR-directed gene synthesis that may be used for all genes, including those with a high G+C content and/or a long sequence. The inventors have discovered three important parameters that play key roles for PCR-directed gene assembly and gene synthesis: (1) the concentration of overlapping oligonucleotides, (2) the type of DNA polymerase used, and (3) the number of PCR amplification cycles. Using a single set of parameters, approximately 20 genes ranging in size between about 300 and about 1700 base pairs with G+C content between about 50% and about 70% were synthesized, demonstrating the general applicability of the methods of the invention for reproducible and successful synthesis of a wide variety of genes.

In one embodiment, the present invention provides a PCR-directed method of synthesizing a gene of interest, wherein the method comprises the steps of (a) determining an optimal concentration of a plurality of overlapping oligonucleotides; (b) assembling the plurality of overlapping oligonucleotides at the determined optimal concentration by at least one cycle of assembly PCR to generate template DNA; and (c) amplifying the template DNA with two separate and distinct outermost overlapping oligonucleotides by at least one cycle of amplification PCR. In another embodiment, the invention provides a PCR-directed method of synthesizing a gene of interest, wherein the method comprises the steps of (a) assembling a plurality of overlapping oligonucleotides by about 5-30 cycles (e.g., about 5-20 cycles) of assembly PCR to generate template DNA; and (b) amplifying the template DNA with two separate and distinct outermost overlapping oligonucleotides by about 10-20 cycles of amplification PCR. In another embodiment, the invention provides a PCR-directed method of synthesizing a gene of interest, wherein the method comprises the steps of (a) assembling a plurality of overlapping oligonucleotides by at least one cycle of assembly PCR to generate template DNA; and (b) amplifying the template DNA with two separate and distinct outermost overlapping oligonucleotides by at least one cycle of amplification PCR, wherein at least one of the steps of assembling the plurality of overlapping oligonucleotides and amplifying the template DNA further comprises the steps of selecting a DNA polymerase and using the selected DNA polymerase, and wherein the DNA polymerase has 3′ to 5′ proofreading activity.

In at least one embodiment, the invention provides a PCR-directed method of synthesizing a gene of interest, wherein the method comprises the steps of (a) determining an optimal concentration of a plurality of overlapping oligonucleotides; (b) assembling the plurality of overlapping oligonucleotides at the determined optimal concentration by about 5-30 cycles (e.g., about 5-20 cycles) of assembly PCR to generate template DNA; and (c) amplifying the template DNA with two separate and distinct outermost overlapping oligonucleotides by about 10-20 cycles of amplification PCR, wherein at least one of the steps of assembling the plurality of overlapping oligonucleotides and amplifying the template DNA further comprises the steps of selecting a DNA polymerase and using the selected DNA polymerase, and wherein the DNA polymerase has a 3′ to 5′ proofreading activity.

In some embodiments, the present invention provides methods of PCR-directed synthesis of genes wherein the optimal concentration of the plurality of overlapping oligonucleotides is in the range of about 0.8 to about 4.0 μM. In another embodiment, the selected DNA polymerase has an error frequency of about 0.01% or less. In another embodiment, the method of the invention further comprises the step of diluting the template DNA after the step of assembling the plurality of overlapping oligonucleotides and prior to the step of amplifying the template DNA.

In some embodiments, the method of the invention further comprises, as a first step, the step of optimizing the plurality of overlapping oligonucleotides. In another embodiment, the step of optimizing the plurality of overlapping oligonucleotides is accomplished using codon-optimization software. In a further embodiment, the step of optimizing the plurality of overlapping oligonucleotides comprises altering the nucleotide sequence of at least one of the plurality of overlapping oligonucleotides such that the nucleotide sequence of the template DNA differs in at least one codon from the nucleotide sequence of the gene of interest. In another further embodiment, the at least one codon of the template DNA is a codon with optimal frequency of usage, and the template DNA encodes a protein having an amino acid sequence identical to the amino acid sequence of the protein encoded by the gene of interest. In another further embodiment, the at least one codon of the template DNA introduces a mutation into the protein encoded by the gene of interest. In some embodiments, the present invention provides methods of PCR-directed synthesis of genes wherein the gene of interest is about 300 to about 1700 base pairs in length.

In at least one embodiment, the present invention provides a nucleic acid molecule synthesized according to the disclosed PCR-directed methods of synthesizing a gene of interest. In another embodiment, the invention provides a vector comprising such a nucleic acid molecule. In another embodiment, the invention provides an expression vector comprising such a nucleic acid molecule operably linked to an expression control sequence. In another embodiment, the invention provides a host cell comprising such an expression vector. In another embodiment, the invention provides a method of producing a polypeptide, comprising the steps of (a) culturing such a host cell under conditions such that the polypeptide is expressed; and (b) purifying the expressed polypeptide from the host cell. In another further embodiment, the invention provides a polypeptide produced by such a method.

### BRIEF DESCRIPTION OF THE DRAWINGS

FIG. 1 is a schematic representation of the general method of PCR-directed gene synthesis comprising the steps of (upper panel) assembly PCR using a plurality of overlapping oligonucleotides (e.g., a, b, c, etc.), wherein the overlapping oligonucleotides are extended using each other as a template to first generate overlapping oligonucleotides (e.g., e, f, g, etc.), and subsequently to generate the template DNA for amplification PCR, and (lower panel) amplification PCR with two outermost overlapping oligonucleotides.

FIG. 2 shows an agarose gel electrophoresis analysis of FAAH (lanes 1a and 1b), hCatSper3 (lanes 2a and 2b), hDAOA (lanes 3a and 3b), pDAO (lanes 4a and 4b), TREM2 (“Trem2”) (lanes 5a and 5b), and GPR55 (lanes 6a and 6b) genes synthesized by 10 cycles of gene assembly PCR followed by 20 cycles of gene amplification PCR (lanes 1a-6a), or by 20 cycles of gene assembly PCR followed by 30 cycles of gene amplification PCR (lanes 1b-6b).

FIG. 3 shows gene synthesis for (FIG. 3A) GPR55, hDAOA, TREM2 (Trem2), FAAH, and (FIG. 3B) IGF1, USAG1, and IGFBP4 genes, assembled and amplified using different DNA polymerases (lanes 1: PRIMESTAR® HS DNA polymerase (PSHS); 2: HIFI® DNA polymerase (HiFi); 3: ACCUPRIME PFX™ DNA polymerase (AccuPrime Pfx); 4: Herculase HS DNA polymerase (Herculase HS); 5: PFUTURBO® HS DNA polymerase (Pfu Turbo HS); and 6: FAILSAFE™ DNA polymerase (FailSafe)) as analyzed by agarose gel electrophoresis. M: MASSRULER™ DNA Ladder Mix.

FIG. 4 shows agarose gel electrophoresis of the PCR products of the GPR55, FAAH, hCatSper3 genes assembled and amplified with PRIMESTAR® HS DNA polymerase and different concentrations (0.8, 2.4, 4.0, and 8.0 μM) of overlapping oligonucleotides.

FIG. 5 demonstrates agarose gel electrophoresis of the mTPH2 gene assembled initially as three fragments (A, B, and C), and subsequently joined to create the full-length gene by splice overlap PCR.

FIG. 6 is a schematic representation of overlap extension PCR, wherein gene fragments A and B are separately generated using outermost overlapping oligonucleotides 1 and 2, and 3 and 4, respectively, and wherein the 5′ portions of outermost overlapping oligonucleotides 2 and 3 are complementary to each other, such that the separately generated fragments A and B are capable of annealing to each other and being extended in a PCR reaction with outermost overlapping oligonucleotides 1 and 4 to generate the full-length gene of interest (as shown by dotted lines).

FIG. 7A shows the codon-optimized sequence of the human DAOA gene (hDAOA) (SEQ ID NO:1); FIG. 7B shows the overlapping oligonucleotides generated by the UpGene program (sense oligonucleotides HDAOAS1-HDAOAS13 (SEQ ID NO:2 to SEQ ID NO:14, respectively) and antisense oligonucleotides HDAOAAS1-HDAOAAS13 (SEQ ID NO:15 to SEQ ID NO:27, respectively)). In the codon-optimized sequence, triplets that have been optimized are shown in uppercase letters (see Gao et al. (2004) supra).

### DETAILED DESCRIPTION

The present invention provides a method of rapidly synthesizing a gene of interest. The method of the present invention is useful in synthesizing any gene of interest; in some embodiments of the invention, the gene of interest may have either (or both) a high G+C content or a long sequence. Additionally, the invention allows for methods of codon optimization that are useful in overcoming poor levels of gene expression of the gene of interest and obtaining high levels of protein for biochemical studies, structural studies, vaccine development, etc. The method of PCR-directed gene synthesis of the present invention may also be useful in other applications, including but not limited to construction of mosaic fusion proteins, construction of linear recombinant DNA, e.g., expression vectors, construction of targeting constructs for gene knockout technology, etc.

### Synthesizing the Gene of Interest

The present invention provides a PCR-directed method of synthesizing a gene of interest, generally comprising the steps of (1) assembling a plurality of overlapping oligonucleotides by at least one cycle of assembly PCR to generate template DNA for the gene of interest, and (2) amplifying the template DNA with two separate and distinct outermost overlapping oligonucleotides in at least one cycle of amplification PCR. The inventors have overcome problems encountered by PCR-directed methods of synthesizing genes in previous published studies by (i) optimizing the concentration of the plurality of overlapping oligonucleotides, (ii) selecting the DNA polymerase to be used in either or both types of PCR (assembly PCR and/or amplification PCR), e.g., high fidelity PRIMESTAR® HS DNA polymerase, and/or (iii) reducing the number of assembly or amplification PCR cycles. Thus, the invention provides a rapid, reproducible, and cost-effective method of synthesizing genes, particularly those that have a long sequence and/or have a high G+C content.

Polymerase chain reaction (PCR) is a method for rapid nucleic acid amplification that is well known in the art (see, e.g., U.S. Pat. Nos. 4,683,195, 4,683,202, and 4,965,188). PCR generally comprises subjecting an oligonucleotide sample, e.g., a sample comprising DNA polymerase, dNTPs, buffer, oligonucleotides, and a template, to at least one cycle comprising the steps of denaturing, annealing (or hybridizing), and elongating (or extending). One skilled in the art will recognize that the denaturing, annealing, and elongating steps of PCR may be effectuated by altering the temperature of the oligonucleotide sample in the presence of the appropriate reagents and polymerase. One of skill in the art will also recognize that the temperatures, the length of time at such temperatures, and the number of PCR cycles that the oligonucleotide sample must be subjected to will differ for different oligonucleotides. Additionally, a skilled artisan will recognize that increased temperature “hot starts” often begin PCR methods, and that a final incubation at about 72° C. may optionally be added to the end of any PCR reaction.

The phrases “PCR-directed gene synthesis,” “PCR-based gene synthesis,” “PCR-directed method of gene synthesis,” or the like, generally comprise the steps of gene assembly and gene amplification. The steps of gene assembly and gene amplification involve the use of PCR, and are referred to herein as “assembly PCR” and “amplification PCR,” respectively.

The PCR-directed method of synthesizing a gene of interest provided by the invention encompasses methods of gene synthesis wherein a plurality of overlapping oligonucleotides are assembled with at least one cycle of assembly PCR to generate template DNA for the gene of interest, and wherein a first and a second outermost overlapping oligonucleotide amplify the template DNA for the gene of interest with at least one cycle of amplification PCR.

A “gene of interest” is a target polynucleotide to be synthesized by a PCR-directed gene synthesis method. In one embodiment of the invention, the gene of interest comprises a gene whose expression is tightly regulated (e.g., through gene copy number, transcriptional control elements, mRNA stability, translational efficiency). In another embodiment of the invention, the gene of interest has a nucleotide sequence with a high G+C content, e.g., at least about 50%, 60%, 70%, or 80% or higher G+C content. In yet another embodiment of the invention, the gene of interest is greater than about 300 bp in length, e.g., greater than about 500 bp in length, greater than about 1000 bp in length, greater than about 1700 bp in length, greater than about 2000 bp in length, greater than about 3000 bp in length, etc.

The gene of interest may be derived from any organism, e.g., plant, animal, protozoan, bacterium, virus, or fungus. The animal from which the gene may be derived may be vertebrate or invertebrate. Examples of vertebrate animals include fish, mammal, cattle, goat, pig, sheep, rodent, hamster, mouse, rat, primate, and human; invertebrate animals include nematodes, other worms, drosophila, and other insects. The gene of interest may also be derived from a cell that is removed, grown, stored or maintained separately from its native environment. The cell may be germ line or somatic, totipotent or pluripotent, dividing or nondividing, parenchymal or epithelial, immortalized or transformed, etc. The cell may be a stem cell or a differentiated cell.

As disclosed herein, a gene of interest synthesized by a method of the present invention is not limited to any type of target gene or nucleotide sequence. For example, the gene of interest need not consist of a full complement of coding, noncoding and regulatory regions, but can comprise a portion or subset of the coding, noncoding and/or regulatory regions. However, the PCR-directed gene synthesis of the following genes is described herein for illustrative purposes: FAAH, hDAOA, pDAO, hCatSper3, GPR55, TREM2, IGF1, USAG1, IGFBP4, and mTPH2.

Nucleic acid hybridization reactions, also referred to as annealing reactions, can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions.

Oligonucleotides, also referred to herein as oligodeoxyribonucleotides or oligoribonucleotides, polynucleotides, or the like, are single-stranded nucleic acid polymers comprising two or more nucleic acids covalently bonded through a sugar-phosphate linkage or the equivalent. An “overlapping oligonucleotide” refers to an oligonucleotide that is complementary to at least a portion of the gene of interest or other polynucleotide, and which provides a free 3′-OH for initiation of DNA synthesis. In the methods of the present invention, overlapping oligonucleotides are capable of initiating DNA synthesis when subjected to at least one cycle of PCR. In particular, during the annealing step of PCR, a first overlapping oligonucleotide may anneal to a second oligonucleotide with a complementary sequence. During the elongation step of PCR, an overlapping oligonucleotide that is annealed to a second oligonucleotide is extended, or elongated, to have and/or consist essentially of a sequence complementary to at least a portion of the sequence of the second oligonucleotide, and preferably complementary to essentially the entire sequence of the second oligonucleotide. In one embodiment of the present invention, the second polynucleotide may be template DNA. In another embodiment, it may be another overlapping oligonucleotide. Thus, each overlapping oligonucleotide preferably hybridizes under stringent conditions to the gene of interest or a fragment thereof and/or at least partially to at least one other overlapping oligonucleotide in the plurality of overlapping oligonucleotides used in the present methods.

A plurality of overlapping oligonucleotides refers to a collection of overlapping oligonucleotides, each of which is complementary to either the sense (+) or the antisense (−) strand of a portion of the gene of interest, such that each oligonucleotide partially hybridizes to at least one other overlapping oligonucleotide, and such that when the overlapping oligonucleotides are assembled by PCR, a template DNA substantially identical, or substantially complementary, to the gene of interest is generated. For example, the methods of the present invention contemplate a template DNA that is at least about: 65%, 70%, 75%, 80%, 85%, 90%, 95%, or 99% or more identical to the gene of interest. In one embodiment of the invention, the plurality of overlapping oligonucleotides contiguously encodes the entire sense and the entire antisense strand of the DNA representing the gene of interest. In another embodiment, the plurality of overlapping oligonucleotides generates a template DNA with a nucleotide sequence that differs by at least one nucleic acid residue from the nucleotide sequence of the gene of interest.

Thus, in one embodiment of the invention, the plurality of overlapping oligonucleotides may be optimized, e.g., for codon optimization, insertion of a specified mutation(s), etc. As one skilled in the art will recognize, each amino acid is encoded by a triplet of nucleotides, otherwise known as a codon. Because the genetic code uses 64 codons to encode 20 amino acids and a stop signal, most amino acids are encoded by more than one codon (degeneracy of the genetic code). Thus, many nucleotide sequences are capable of encoding the same protein. Different codons are used with different frequencies, and these frequencies are directly correlated to the concentration of corresponding transfer RNA. Additionally, genes that encode infrequently used codons are generally expressed at low levels, i.e., have low levels of protein expression. A “codon with optimal frequency of usage” refers to a nucleotide triplet used most commonly by an organism to encode a particular amino acid. Therefore, one skilled in the art will recognize that in order to maximize expression of the synthesized gene, the plurality of overlapping oligonucleotides may be optimized by altering the nucleotide sequence of at least one overlapping oligonucleotide of the plurality of overlapping oligonucleotides such that the sequence of the template DNA generated by assembling the optimized plurality of overlapping oligonucleotides differs from the sequence of the gene of interest due to the introduction of replacement codons (e.g., a different codon, based on the degeneracy of the genetic code, coding for the same amino acid). In another embodiment of the invention, the plurality of overlapping oligonucleotides is optimized such that the template DNA resulting from assembling the optimized plurality of overlapping oligonucleotides encodes the same amino acid sequence as the gene of interest but differs in at least one codon from the nucleotide sequence of the gene of interest, wherein the at least one codon of the template DNA is the codon with optimal frequency of usage.

In addition to optimizing the plurality of overlapping oligonucleotides to generate template DNA comprising at least one codon with optimal frequency of usage, the nucleotide sequence of at least one of the plurality of overlapping oligonucleotides may be optimized to introduce at least one mutation into the protein encoded by the template DNA, e.g., wherein at least one codon is replaced with a codon that encodes a different amino acid. Alternatively, other sequences (e.g., restriction enzymes sites or regulatory sequences such as the Kozak sequence, the Shine-Delgarno sequence, etc.) may be introduced into some of the plurality of overlapping oligonucleotides, e.g., the two outermost overlapping oligonucleotides, to facilitate subsequent cloning and expression.

Furthermore, certain genes contain sequences associated with a high degree of mRNA instability (Maurer et al. (1999) Nucleic Acids Res. 27:1664-73; Rabbits et al. (1985) EMBO J. 4:3727-33). In some embodiments of the present invention, the nucleotide sequence of at least one of the plurality of overlapping oligonucleotides may be optimized to improve mRNA stability of the synthesized gene. One of ordinary skill in the art can readily determine which of the plurality of overlapping oligonucleotides may be optimized to accomplish such improved mRNA stability. In addition, the nucleotide sequence of at least one of the plurality of overlapping nucleotides may be optimized to insert or delete restriction enzyme sites in the gene of interest, to minimize mRNA secondary structure, to delete cryptic splice sites, etc.

Methods of optimizing a plurality of overlapping oligonucleotides may be accomplished manually or with the use of computer software. Such software is well known in the art, and includes but is not limited to: UpGene (University of Pittsburgh, Pa.), DNAWorks (National Cancer Institute, MD; see, e.g., mcl1.ncifcrf.gov/lubkowski), GMAP (National Institute of Medical Research, Chandigarh, India), COD OP (National Institute of Medical Research, London, UK), Prot2DNA (DNA2.0 Inc., CA), GeMS (Kosan Biosciences, CA), JCat (Technische Universitat Braunschweig, Braunschweig, Germany), Synthetic Gene Designer (DNA2.0 Inc., CA), DNA Builder (Pacific Northwest National Laboratory, WA), Gene Composer (Emerald Biosystems, WA), GeneDesign (John Hopkins University, MD), or any other software with codon optimization capabilities.

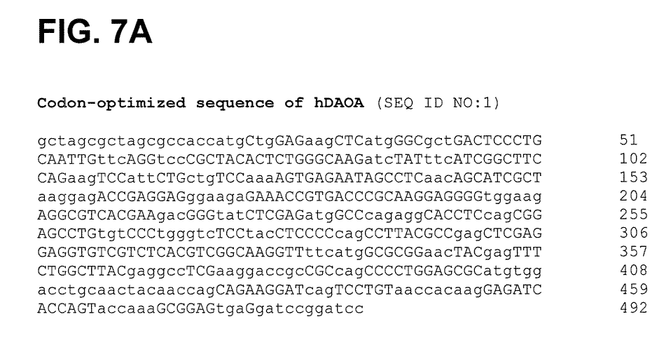

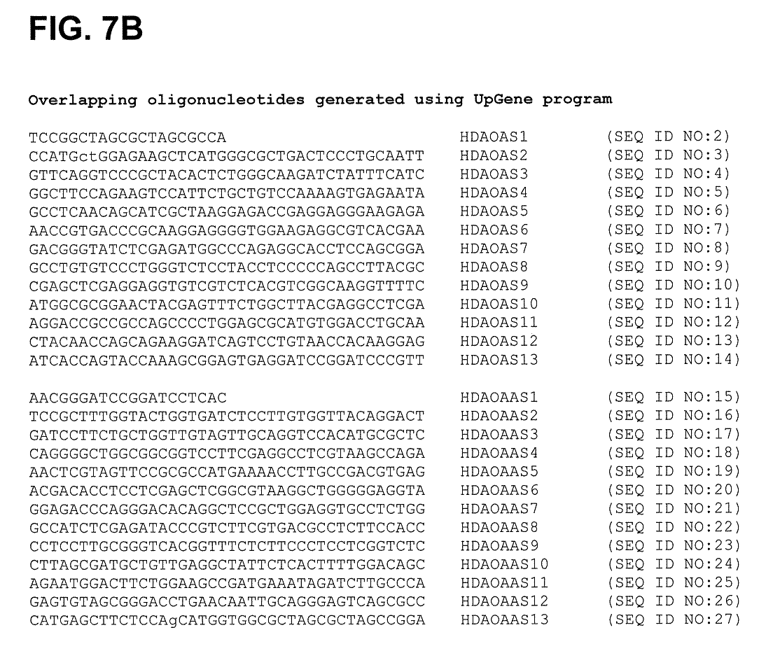

In one embodiment of the present invention, the plurality of overlapping oligonucleotides may be optimized using the UpGene computer software (www.vectorcore.pitt.edu/upgene). The graphical user interface of UpGene consists of the input window, where a user specifies a sequence, e.g., amino acid or nucleotide sequence of the gene of interest (e.g., in the organism(s) of choice), and any other modifications necessary, e.g., restriction enzyme sequences to be added at 3′ and 5′ ends. The program allows the user to optimize codons for higher levels of expression of the gene of interest and/or introduce mutations into oligonucleotides that would result in at least one altered amino acid. Based on the sequence in the input window and other requirements specified by the user, the output window presents a plurality of overlapping oligonucleotides, which include the internal overlapping oligonucleotides and two distinct outermost overlapping oligonucleotides (see, e.g., FIG. 7B).

The phrases “two separate and distinct outermost overlapping oligonucleotides,” “two distinct outermost overlapping oligonucleotides,” or “two outermost overlapping oligonucleotides” refer to the flanking 5′ sense and the flanking 5′ antisense overlapping oligonucleotides. The flanking 5′ sense and 5′ antisense overlapping oligonucleotides are complementary to the sequences on either end of the gene of interest. The term “internal overlapping oligonucleotides” refers to all overlapping oligonucleotides in the plurality of overlapping oligonucleotides other than the two outermost overlapping oligonucleotides.

Generally, an overlapping oligonucleotide may be about 20-45 base pairs (“bp”) in length (e.g., about 25-40 bp). In one embodiment of the invention, the two outermost overlapping oligonucleotides are about 25 bp in length. In another embodiment of the invention, the internal overlapping oligonucleotides are about 40 bp in length. An overlapping oligonucleotide in a plurality of overlapping oligonucleotides may overlap, i.e., be complementary to, all or a portion of one or more other overlapping oligonucleotide(s). In one embodiment, an overlapping oligonucleotide overlaps another by about 20 bp.

A skilled artisan will recognize that an overlapping oligonucleotide may be chemically synthesized or purchased. Furthermore, it is known that overlapping oligonucleotides may be purchased in lyophilized form, and subsequently resuspended by the user in sterile water, preferably DNase- and/or RNase-free water, at a desired concentration, including an initial (or stock) concentration, e.g., about 10 to about 50 μM (e.g., about 20 to about 40 μM), which solution can then be diluted to a final desired concentration, e.g., in a 1:10 dilution. Alternatively, overlapping oligonucleotides may also be purchased prediluted in water in 96-well microtiter plates at a predetermined concentration and volume.

The inventors overcame the general inapplicability of PCR-directed methods of gene synthesis by discovering that the concentration of the plurality of overlapping oligonucleotides is critical. Thus, in one embodiment of the invention, the plurality of overlapping oligonucleotides is diluted to a concentration and volume that is optimized for the gene of interest. The optimal concentration of the plurality of overlapping oligonucleotides will vary for different PCR reactions and can be experimentally determined using a set of PCR-directed gene synthesis reactions with varying concentrations of the plurality of overlapping oligonucleotides (see, e.g., Example 5). One of skill in the art will recognize that the optimal concentration of the overlapping oligonucleotides is the concentration that allows successful optimized synthesis of the gene of interest. In one embodiment of the invention, the optimal concentration of overlapping oligonucleotides refers to a concentration that allows the synthesis of the greatest number of molecules of the gene of interest when all other conditions are held the same. In another embodiment of the invention, the optimal concentration of overlapping oligonucleotides is in the range of about 0.8 to about 4.0 μM.

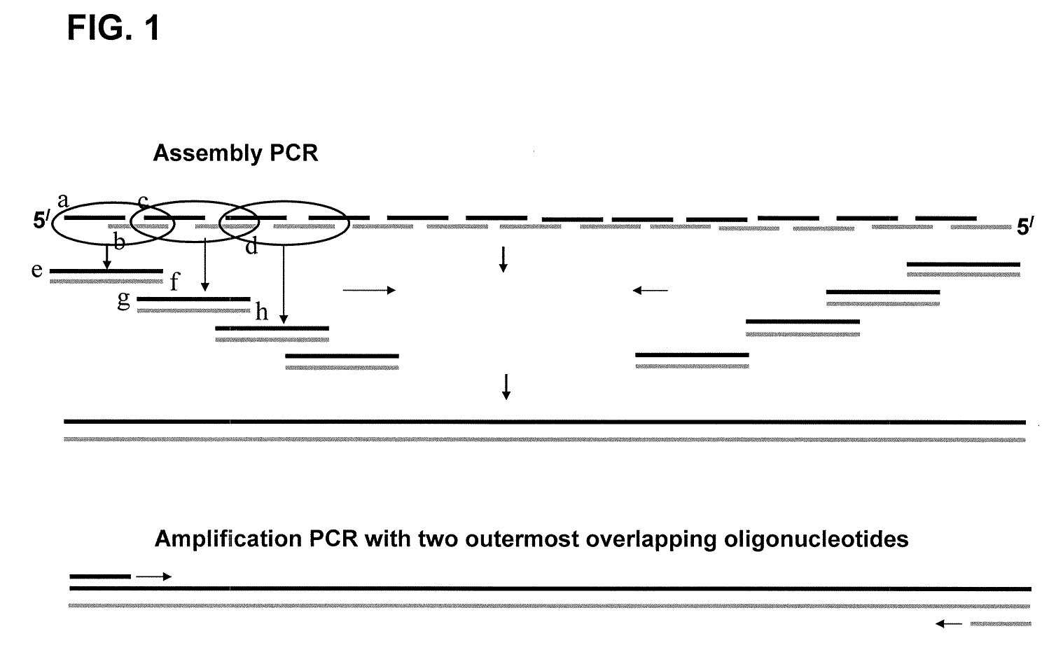

“Assembly PCR” refers to the step(s) in PCR-directed gene synthesis wherein an oligonucleotide sample comprising dNTPs, DNA polymerase, buffer, and a plurality of overlapping oligonucleotides is subjected to at least one cycle of PCR, e.g., less than about 55 cycles, less than about 30 cycles, less than about 20 cycles, or about 5-20 cycles of PCR. In general, each cycle of PCR results in elongation of the synthesized sequence by incorporation of at least one overlapping oligonucleotide from the plurality of overlapping oligonucleotides. For example, in the upper panel of FIG. 1, overlapping oligonucleotides “a” and “b” are subjected to assembly PCR to synthesize sequences “e” and “f.” This process continues until template DNA comprising a sequence substantially identical to the complete sequence of the gene of interest is generated. Assembly PCR may comprise a denaturing step at about 95-98° C., for a period of about 20-45 seconds; an annealing step at about 50° C. for a period of about 30-45 seconds; and an elongating step at about 72° C. for a period of about 30 seconds.

As described herein, the phrase “template DNA” refers to the polynucleotide generated as a result of assembly PCR that may be used as a “template” in the step of amplification PCR.

“Amplification PCR” herein refers to a step(s) in PCR-directed gene synthesis wherein the template DNA generated as a result of assembly PCR is amplified exponentially with the two separate and distinct outermost overlapping oligonucleotides of the plurality of overlapping oligonucleotides, until desired amounts of DNA are generated. Amplification PCR comprises at least one cycle of PCR, e.g., less than about 25 cycles, less than about 20 cycles, or about 10-20 cycles of PCR. In one embodiment of the invention, the step of amplification PCR comprises a denaturing step of about 95-98° C., for a period of about 20-45 seconds; an annealing step of about 50° C. for a period of about 30-45 seconds; and an elongating step of about 72° C. for a period of about 60 seconds per every 1000 bp of DNA being amplified.

The inventors have discovered that the particular DNA polymerase used in either or both assembly and amplification PCR is critical for the synthesis of some genes. Thus, in one embodiment of the invention, the method further comprises selecting a DNA polymerase to be used to synthesize a gene of interest. In one embodiment of the invention, the DNA polymerase selected is the DNA polymerase that allows the synthesis of the greatest number of molecules of the gene of interest when all other conditions are held the same. Preferably, the selected DNA polymerase is capable of 3′ to 5′ proofreading activity such that nucleotide mismatching and false initiation of DNA synthesis are reduced or prevented.

“3′ to 5′ proofreading ability,” also known in the art as “3′ to 5′ exonuclease activity,” is the ability of some DNA polymerases, e.g., Taq DNA polymerase, PRIMESTAR® HS DNA polymerase, etc., to recognize errors in nucleotides incorporated into an elongating polynucleotide sequence, and to remove such errors. DNA polymerases having such 3′ to 5′ proofreading ability are known as “high fidelity DNA polymerases.” One skilled in the art will recognize that various high fidelity DNA polymerases differ in their ability to remove mismatched nucleotides from the elongating DNA molecule.

Nonlimiting examples of DNA polymerases that may be used with the methods of the invention include HIFI® DNA polymerase (Invitrogen, Carlsbad, Calif.), ACCUPRIME PFX™ DNA polymerase (Invitrogen), Herculase HS DNA (Stratagene, LaJolla, Calif.), PFUTURBO® HS DNA polymerase (Stratagene), FAILSAFE™ DNA polymerase (Epicentre, Madison, Wis.), etc. In one embodiment of the present invention, the selected DNA polymerase is the PRIMESTAR® HS DNA polymerase (Takara Mirus Bio, Inc., Madison, Wis.), which has an error frequency of about 0.0048% (product insert, Takara Mirus Bio, Inc.; see also Takara Mirus Bio, Inc. website at www.takaramirusbio.com). The estimated error frequency is based on general use of the polymerase in PCR procedures, as determined by standard measurements known to those of ordinary skill in the art (see, e.g., www.takaramirusbio.com). In another embodiment of the invention, the DNA polymerase is one with an error frequency of less than about 0.01%, less than about 0.009%, less than about 0.008%, less than about 0.007%, less than about 0.006%, or less than about 0.005%. Such low error frequencies are achieved due to the robust 3′ to 5′ proofreading ability of the polymerase.

The inventors further identified that the number of PCR cycles for assembly and amplification PCR may be reduced. Consequently, in one embodiment of the invention, a gene of interest is synthesized by as few as 5-10 cycles of assembly PCR followed by as few as 10-15 cycles of amplification PCR, e.g., using PRIMESTAR® HS DNA polymerase. This is a significant improvement over previous published studies, which teach the use of about 25-55 cycles of PCR for gene assembly and about 23-25 cycles of PCR for gene amplification. The ability to synthesize genes from a plurality of overlapping oligonucleotides in a reduced number of PCR cycles minimizes both the time required to obtain a synthetic gene, as well as the rate of errors introduced during PCR amplification.

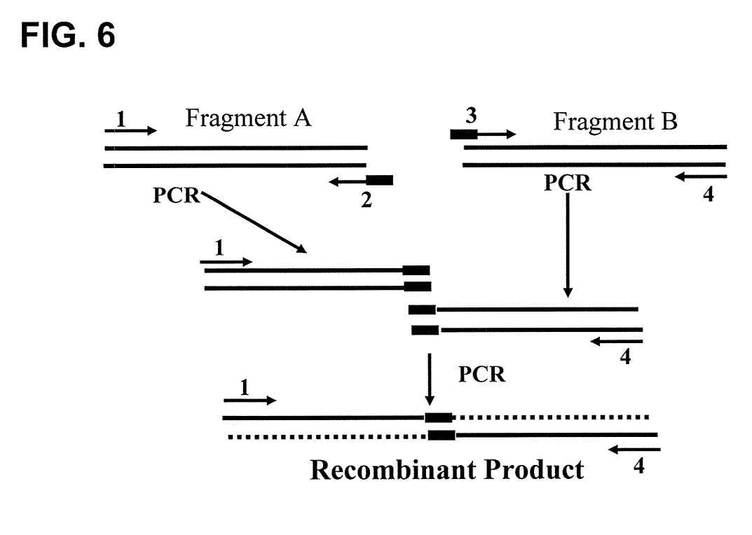

Longer or other difficult-to-synthesize genes may be synthesized by block or “fragment” combination. The method of PCR-directed gene synthesis by block combination encompasses dividing the gene of interest into several overlapping partial gene fragments, and synthesizing each of these fragments according to the methods of the present invention. Thereafter, the full-length gene may be obtained by combining the gene fragments in overlap extension PCR. As used herein, “splice overlap PCR” or “overlap extension PCR” refer to a gene assembly method wherein the gene of interest is divided into two or more gene fragments, and wherein the gene fragments are first separately generated, e.g., using the PCR-directed gene synthesis method of the present invention with a plurality of overlapping oligonucleotides. For example, in FIG. 6, fragments A and B are first separately generated using outermost overlapping oligonucleotides 1 and 2, and 3 and 4, respectively. Outermost overlapping oligonucleotides 2 and 3 are designed in such a way that they are complementary to their corresponding genes in the 3′ portions, while their 5′ portions are complementary to each other. Thus, when fragments A and B are separately generated, the 3′ end of the sense strand of fragment A is complementary to the 3′ end of the antisense strand of fragment B. In overlap extension PCR, the partial overlap of fragments A and B allows outermost overlapping oligonucleotides 1 and 4 to be extended such that a nucleotide sequence comprising both fragment A and fragment B is generated. The method of overlap extension PCR is described in detail in Horton et al. (1989) Gene 77:61-68.

### Cloning and Expressing the Synthesized Gene of Interest

The sequence of a gene synthesized according to the method of the invention may be confirmed by polynucleotide sequencing. A gene of interest synthesized according to the methods of the invention may be cloned and/or ligated into a vector of choice. Additionally, a gene of interest synthesized according to the methods of the present invention may be operably linked to an expression control sequence and cloned and/or ligated into an expression vector for recombinant expression of the gene of interest. General methods of both sequencing and expressing polynucleotides are well known in the art.

As is well known, expression of the gene of interest involves, inter alia, transcribing the polynucleotides into RNA, which may or may not then be translated into protein. Expression of genes refers to an observable increase in the level of the products of the gene of interest (e.g., RNA and/or protein), and may be detected by examination of the outward properties of the host cell or organism, or by biochemical techniques such as hybridization reactions (e.g., Northern blot analysis, RNase protection assays, microarray analysis, etc.), reverse transcription and polymerase chain reactions, binding reactions (e.g., Western blots, ELISA, FACS, etc.), reporter assays, drug resistance assays, etc.

An expression vector, as used herein, is intended to refer to a nucleic acid molecule capable of transporting another nucleic acid molecule (e.g., a polynucleotide, e.g., a gene of interest) to which it has been linked into a host cell or cell-free system, and allowing expression of the transported nucleic acid molecule. One type of expression vector is a plasmid, which refers to a circular double stranded DNA into which additional DNA segments may be ligated. Another type of expression vector is a viral vector, wherein additional DNA segments may be ligated into a viral genome. Certain vectors are capable of autonomous replication in a host cell into which they are introduced (e.g., bacterial vectors having a bacterial origin of replication, and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of the other polynucleotide (e.g., the gene of interest) to which they are operably linked. Such vectors are referred to herein as recombinant expression vectors (or simply, expression vectors). In general, expression vectors of utility in recombinant DNA techniques are often in the form of plasmids. In the present specification, plasmid and vector may be used interchangeably as the plasmid is the most commonly used form of vector. However, the invention is intended to include other forms of expression vectors, such as viral vectors (e.g., replication defective retroviruses, adenoviruses and adeno-associated viruses) that serve equivalent functions.

A number of cell lines may act as suitable host cells for recombinant expression of the genes synthesized according to the methods of the present invention. Mammalian host cell lines include, for example, COS cells, CHO cells, 293T cells, A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants.

Alternatively, it may be possible to recombinantly express genes synthesized according to the methods of the present invention in lower eukaryotes such as yeast, or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the genes synthesized according to the methods of the present invention results in production of corresponding polypeptides in yeast or bacteria, it may be necessary to modify them by, for example, phosphorylation or glycosylation of appropriate sites, in order to obtain functionality. Such covalent attachments may be accomplished using well-known chemical or enzymatic methods.

Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment that allows correct formation of disulfide bonds (a redox system). General methods of refolding are disclosed in Kohno (1990) Meth. Enzymol 185:187-95. U.S. Pat. No. 5,399,677 and EP 0433225 describe other appropriate methods.

The polypeptides encoded by the genes synthesized according to the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MAXBAC® kit, Invitrogen, Carlsbad, Calif.).

The polypeptides encoded by the genes synthesized according to the methods of the present invention may be prepared by growing a culture of transformed host cells under culture conditions necessary to express the desired protein. Following recombinant expression in the appropriate host cells, the polypeptides of the present invention may then be purified from culture medium or cell extracts using known purification processes, such as gel filtration and ion exchange chromatography. Soluble polypeptides can be purified from conditioned media. Membrane-bound polypeptides can be purified by preparing a total membrane fraction from the expressing cell and extracting the membranes with a nonionic detergent such as Triton X-100. Purification may also include affinity chromatography with agents known to bind the polypeptides of the present invention. These purification processes may also be used to purify the polypeptides encoded by the genes of interest from other sources, including natural sources. The polypeptides encoded by genes synthesized according to the method of the present invention may also be expressed as a product of transgenic animals, e.g., as a component of the milk of transgenic cows, goats, pigs, or sheep, which are characterized by somatic or germ cells containing a polynucleotide sequence synthesized according to the methods of the present invention.

The methods that may be used to purify polypeptides encoded by the genes synthesized according to the present invention are known to those skilled in the art. For example, a polypeptide of the invention may be concentrated using a commercially available protein concentration filter, for example, an Amicon or Millipore Pellicon ultrafiltration unit (Millipore, Billerica, Mass.). Following the concentration step, the concentrate can be applied to a purification matrix such as a gel filtration medium. Alternatively, an anion exchange resin can be employed, for example, a matrix or substrate having pendant diethylaminoethyl (DEAE) or polyethyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred (e.g., S-SEPHAROSE® columns). The purification of polypeptides from culture supernatant may also include one or more column steps over such affinity resins as concanavalin A-agarose, HEPARIN-TOYOPEARL® or CIBACROM BLUE 3GA SEPHAROSE®; or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the polypeptides. Affinity columns including antibodies to the polypeptides can also be used in purification steps in accordance with known methods. Some or all of the foregoing purification steps, in various combinations or with other known methods, can also be employed to provide a substantially purified isolated recombinant protein.

Alternatively, the polypeptides encoded by genes synthesized according to the methods of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen, respectively. Polypeptides can also be tagged with an epitope and subsequently identified or purified using a specific antibody to the epitope. A preferred epitope is the FLAG epitope, which is commercially available from Eastman Kodak (New Haven, Conn.).

Even though the invention has been described with a certain degree of particularity, it is evident that many alternatives, modifications, and variations will be apparent to those skilled in the art in light of the disclosure. Accordingly, it is intended that all such alternatives, modifications, and variations, which fall within the spirit and scope of the invention, be embraced by the defined claims.

The entire contents of all references, patents, and patent applications cited throughout this application are hereby incorporated by reference herein.

The Examples which follow are set forth to aid in the understanding of the invention but are not intended to, and should not be construed to, limit its scope in any way. The Examples do not include detailed descriptions of conventional methods, such as PCR and gel electrophoresis, or those methods employed in the construction of vectors, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art.

### EXAMPLES

Embodiments of the invention are discussed herein. The general methods of PCR-directed gene synthesis from the plurality of overlapping oligonucleotides are described in Example 2. The method of optimizing the number of assembly and amplification PCR cycles is described in Example 3. The method of selecting the type of DNA polymerase to be used is found in Example 4, and the method of optimizing oligonucleotide concentration is found in Example 5. “Block combination” gene synthesis and gene mutation by PCR-directed gene synthesis methods of the invention are described in Examples 6 and 7, respectively.

### Example 1

### Materials and Methods

### Example 1.1

### DNA Polymerases

The following DNA polymerases were used as indicated: PRIMESTAR® HS DNA polymerase (Takara Mirus Bio, Inc.), HIFI® DNA polymerase (Invitrogen), ACCUPRIME PFX™ DNA polymerase (Invitrogen), Herculase HS DNA polymerase (Stratagene), PFUTURBO® HS DNA polymerase (Stratagene), FAILSAFE™ DNA polymerase (Epicentre, Madison, Wis.).

### Example 1.2

### Genes of Interest

The following genes of interest were used (and the corresponding GenBank accession record locators for their nucleotide sequences are included): FAAH (Goparaju et al. (1999) Biochim. Biophys. Acta 1441:77-84; GenBank Acc. No. NM_{—}213914), hDAOA (Chumakov et al. (2002) Proc. Natl. Acad. Sci. USA 99:13675-80; GenBank Acc. No. NM_{—}172370; see also codon-optimized sequence and overlapping oligonucleotides in FIGS. 7A and 7B), pDAO (Fukui et al. (1987) Biochemistry 26:3612-18; GenBank Acc. No. NM_{—}214066), hCatSper3 (Lobely et al. (2003) Reprod. Biol. Endocrinol. 1:53; GenBank Acc. No. BC101692), GPR55 (Sawzdargo et al. (1999) Mol. Brain. Res. 64:193-98; GenBank Acc. No. NM_{—}005683), TREM2 (Daws et al. (2001) Eur. J. Immunol. 31:783-91; GenBank Acc. No. NM_{—}018965), IGF1 (Jansen et al. (1983) Nature 306:609-11; GenBank Acc. No. NM_{—}000618), USAG1 (Laurikkala et al. (2003) Dev. Biol. 264:91-105; GenBank Acc. No. AB059270), IGFBP4 (LaTour et al. (1990) Mol. Endocrinol. 4:1806-14; GenBank Acc. No. NM_{—}001552), and mTPH2 (Walther et al. (2003) Science 299:76; GenBank Acc. No. NP_{—}775567).

### Example 1.3

### DNA Markers

DNA molecular weight markers were either MASSRULER™ DNA Ladder Mix (Fermentas, Hanover, Md.), or 1 kb Plus DNA Ladder (Invitrogen).

### Example 1.4

### Overlapping Oligonucleotide Design

A plurality of overlapping oligonucleotides for each gene of interest was designed using the publicly available Web-based DNA codon optimization algorithm, UpGene (www.vectorcore.pitt.edu/upgene), and purchased from either Invitrogen or Integrated DNA Technologies (Coralville, Iowa).

### Example 2

### PCR-Directed Gene Synthesis

### Example 2.1

### General PCR-Directed Gene Synthesis

The assembly PCR step is schematically shown in FIG. 1 (upper panel). The single-stranded end of a first overlapping oligonucleotide (“a”) that is complementary to the single-stranded end of a second overlapping oligonucleotide (“b”) is extended/elongated from 5′ to 3′ direction with DNA polymerase during the first assembly PCR cycle to form oligonucleotides “e” and “f.” Similarly, overlapping oligonucleotides “c” and “d” are extended/elongated using each other as template to form oligonucleotides “g” and “h,” and so on. During the next cycle, “g” and “h” are similarly extended, resulting in increasingly larger DNA fragments until a full-length template DNA is obtained.

After assembly of the plurality of overlapping oligonucleotides to generate template DNA, amplification PCR is performed, as shown in the lower panel of FIG. 1. Amplification PCR uses two distinct outermost overlapping oligonucleotides to amplify the full-length template DNA generated as a result of assembly PCR.

### Example 2.2

### PCR-Directed Gene Synthesis

Equal volumes of each overlapping oligonucleotide (designed by the UpGene algorithm and purchased from either Invitrogen or Integrated DNA Technologies), at an initial concentration of either 20 μM (for FAAH only) or 40 μM, were mixed to form a plurality of overlapping oligonucleotides. The plurality of overlapping oligonucleotides were diluted 10-fold into a 50 μl final volume of the assembly PCR reaction (e.g., 2 mM dNTPs and 1.25 units of PRIMESTAR® HS DNA polymerase in 1×PCR buffer). For other DNA polymerases, appropriate amounts of the polymerase, dNTPs, and buffer, according to manufacturer's instructions, were used.

Assembly of the overlapping oligonucleotides was carried out in a 0.2 ml sterile thin-walled PCR tube. Assembly PCR was initiated with a 4-minute denaturing step of 95° C. (i.e., “hot start”), followed by 5-20 cycles of a denaturing step of 95° C. for 30 seconds, an annealing step of 50° C. for 30-45 seconds, and an elongating step of 72° C. for 30 seconds. The last step in the protocol was an incubation cycle at 72° C. for 5 minutes.

For assembly PCR of genes with a high G+C content, the assembly PCR reaction protocol consisted of a denaturing step of, e.g., 30 seconds to 4 minutes at 98° C. (i.e., “hot start”), an annealing step of 50° C. for 30-45 seconds, and an elongating step of 72° C. for 30-60 seconds. Subsequent cycles (ranging from 5 to 30 cycles) of assembly PCR consisted of a denaturing step of 98° C. for 20-30 seconds, an annealing step of 50° C. for 30-45 seconds, and an elongating step of 72° C. for 30-60 seconds. The last step was a 5-minute 72° C. incubation step.

For gene amplification PCR, the template DNA was diluted at least 10-fold into a 50 μl oligonucleotide sample comprising 2 mM dNTP, 1.25 units DNA polymerase, 1×PCR buffer, and 1.0-1.5 μM of each outermost overlapping oligonucleotides. The protocol for gene amplification PCR was essentially the same as the protocol of the gene assembly PCR with the exception of the elongating time, which was adjusted according to the size of the gene being amplified (60 seconds per 1000 base pairs of DNA being generated), and the number of amplification cycles, which ranged between 10 and 20 cycles. For amplification PCR of genes of interest with high G+C base pair content, the denaturing steps were performed at 98° C.

### Example 2.3

### Cloning and Sequencing of Gene Synthesis Products

All synthetic DNA generated by PCR-directed gene synthesis were purified using a Qiagen PCR purification kit (Qiagen, Valencia, Calif.) and then ligated into either PCR-blunt vector using ZERO BLUNT PCR® cloning kit (Invitrogen) or other vectors of choice. The ligation reaction was carried out at 15° C. for 2-4 hours, and then used to transform TOP10 chemically competent cells (Invitrogen) according to the manufacturer's suggested protocol. The resulting clones were selected on LB agar plates containing appropriate antibiotic. Due to the presence of the ccdB gene in PCR-blunt vector, more than 80% of the screened transformed colonies contained the correct size inserts as verified by DNA sequencing.

### Example 3

### Effect of Assembly PCR Cycles and Amplification PCR Cycles on PCR-Directed Gene Synthesis

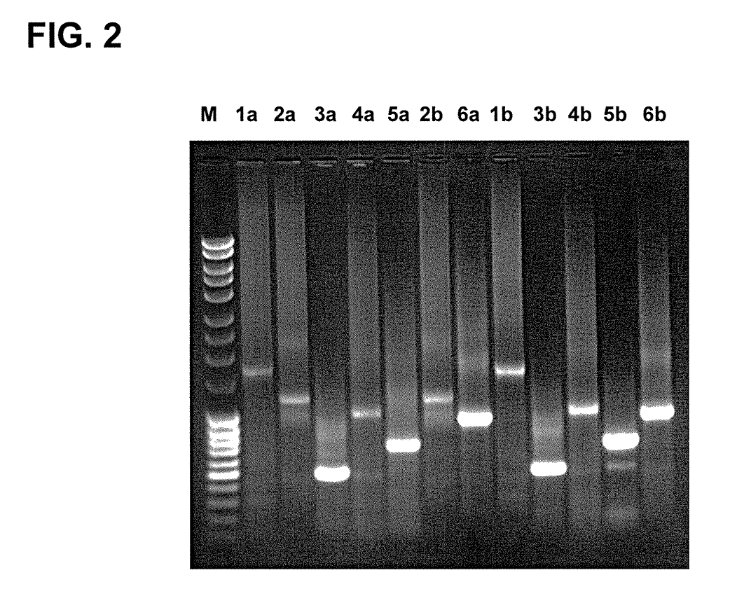

The minimum numbers of assembly PCR cycles and amplification PCR cycles required for successful gene synthesis with PRIMESTAR® HS DNA polymerase were determined using the methods described in Example 2. In contrast to previous reports (Stemmer et al. (1995) supra; Hoover and Lubkowski (2002) supra; and Gao et al. (2004) supra), successful and reproducible PCR-directed gene synthesis of genes of interest required a combination of a minimum of 5-10 cycles of gene assembly PCR followed by 10-20 cycles of gene amplification PCR, e.g., 10 cycles of gene assembly PCR followed by 20 cycles of gene amplification PCR was sufficient for gene synthesis (FIG. 2, lanes 1a-6a).

### Example 4

### Effect of DNA Polymerases on PCR-Directed Gene Synthesis

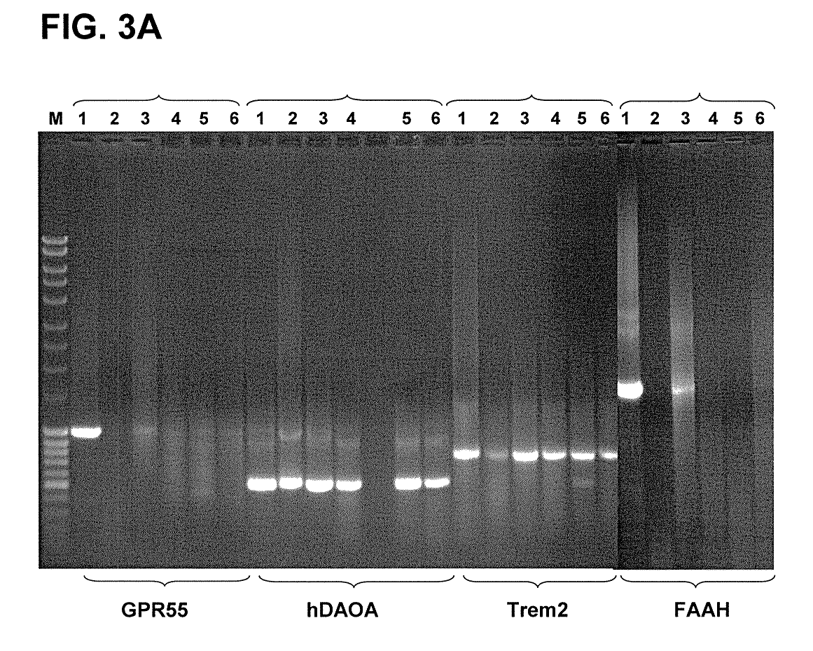

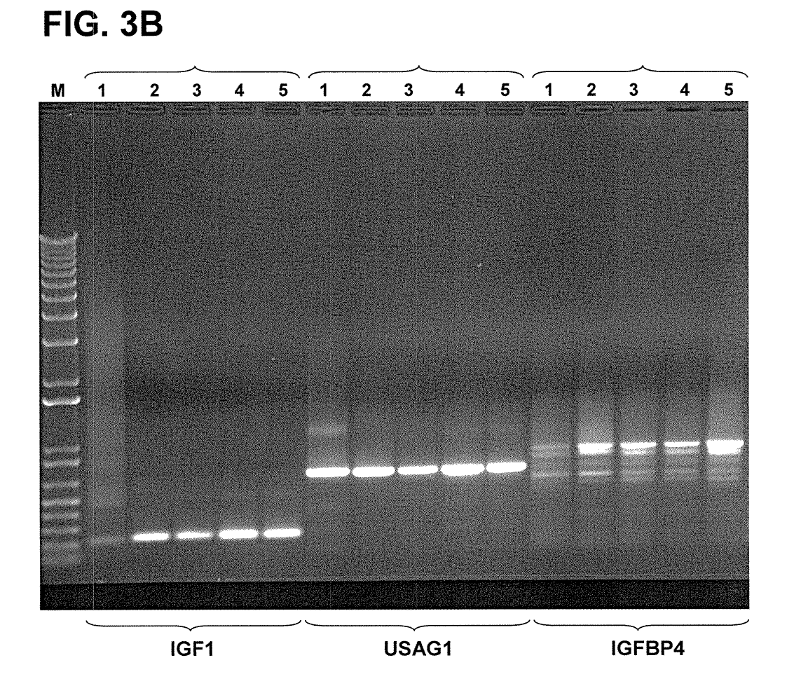

Several DNA polymerases with 3′ to 5′ proofreading ability were tested to determine which DNA polymerase is able to synthesize the most genes of variable length and G+C content. DNA polymerases such as PRIMESTAR® HS, HIFI®, ACCUPRIME PFX™, Herculase HS, PFUTURBO® HS, and FAILSAFE™ were tested for PCR-directed gene synthesis of GPR55, hDAOA, TREM2, and FAAH genes (FIG. 3A), as well as IGF1, USAG1, and IGFBP4 genes (FIG. 3B). PCR-directed gene synthesis was performed with 10 cycles of assembly PCR followed by 20 cycles of amplification PCR. Surprisingly, some of the genes tested, e.g., FAAH, and GPR55, were only successfully synthesized with PRIMESTAR™ HS (PSHS) DNA polymerase (lane 1). This finding highlights the importance of selecting an appropriate DNA polymerase for reproducible and successful PCR-directed gene synthesis.

### Example 5

### Determining the Optimal Concentration of the Plurality of Overlapping Oligonucleotides for PCR-Directed Gene Synthesis

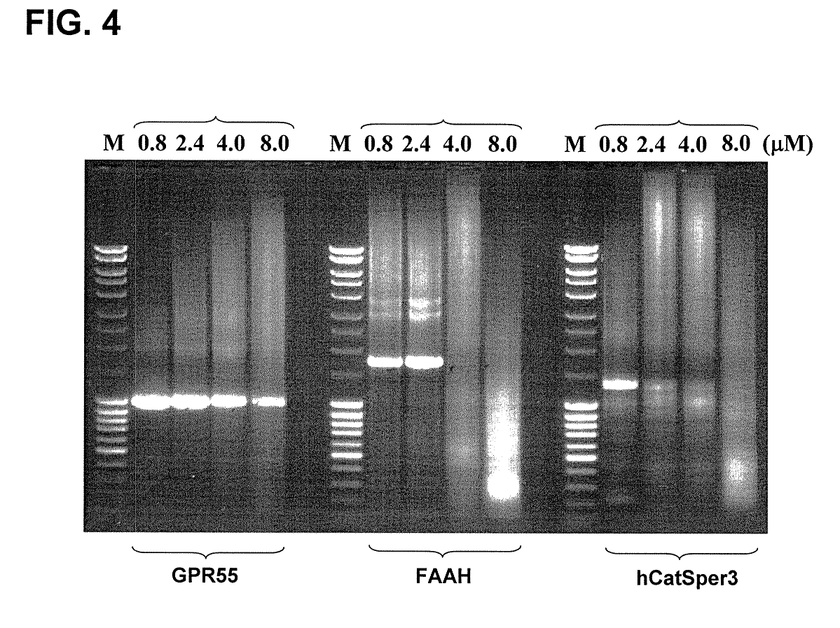

To determine the optimal concentration of the plurality of overlapping oligonucleotides for PCR-directed gene synthesis, GPR55, FAAH, and hCatSper3 genes were synthesized according to the methods of Example 2 in the presence of different concentrations of the plurality of overlapping oligonucleotides, i.e., 0.8, 2.4, 4.0, and 8.0 μM. While GPR55 was successfully synthesized in the presence of all concentrations of the plurality of overlapping oligonucleotides tested, the optimal concentration of the plurality of overlapping oligonucleotides for FAAH was either 0.8 or 2.4 μM, and for hCatSper3 was only 0.8 μM (FIG. 4). This finding suggests that the concentration of the plurality of overlapping oligonucleotides is critical for the success of PCR-directed gene synthesis, and should be determined for each gene of interest through routine experimentation.

### Example 6

### Gene Synthesis by Block Combination

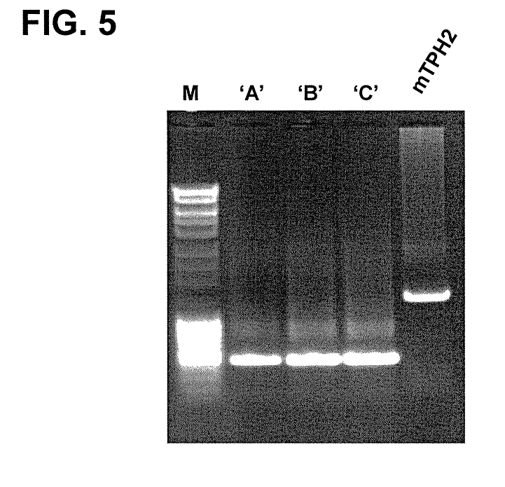

Some genes, e.g., mTPH2, could not be synthesized using a PCR-directed gene synthesis method merely comprising gene assembly PCR from a plurality of overlapping oligonucleotides followed by gene amplification PCR. Synthesis of mTPH2 involved PCR-directed gene synthesis of three overlapping partial gene fragments, or blocks, A, B, and C (FIG. 5), all of roughly equal size. The full-length mTPH2 gene was then obtained by combining equal amounts of the three blocks, subjecting them to overlap extension PCR, wherein the fragments were denatured, reannealed, and subsequently amplified with outermost overlapping oligonucleotides at a concentration of 1.5 μM.

### Example 7

### Gene Mutation by PCR-Directed Gene Synthesis with Altered Oligonucleotides

A method of PCR-directed gene synthesis from a plurality of overlapping oligonucleotides was used to introduce desired mutations into the TREM2 gene. The TREM2 gene with 29 simultaneous mutations was generated by substituting mutant oligonucleotides for wild-type TREM2-based oligonucleotides at the desired locations. The mutant TREM2 gene was fully sequenced to verify the presence of the 29 mutations.

We claim:

1. A PCR-directed method of synthesizing a gene of interest, wherein the method comprises the following steps:
(a) determining an optimal concentration of a plurality of overlapping oligonucleotides;
(b) assembling the plurality of overlapping oligonucleotides at the determined optimal concentration by at least one cycle of assembly PCR to generate template DNA; and
(c) amplifying the template DNA with two separate and distinct outermost overlapping oligonucleotides by at least one cycle of amplification PCR.

2. The method of claim 1, wherein the at least one cycle of assembly PCR is about 5 to about 20 cycles, and wherein the at least one cycle of amplification PCR is about 10 to about 20 cycles.

3. The method of claim 1, wherein at least one of the steps of assembling the plurality of overlapping oligonucleotides and amplifying the template DNA further comprises the steps of selecting a DNA polymerase and using the selected DNA polymerase, and wherein the DNA polymerase has a 3′ to 5′ proofreading activity.

4. The method of claim 2, wherein at least one of the steps of assembling the plurality of overlapping oligonucleotides and amplifying the template DNA further comprises the steps of selecting a DNA polymerase and using the selected DNA polymerase, and wherein the DNA polymerase has a 3′ to 5′ proofreading activity.

5. The method of claim 1, wherein the optimal concentration of the plurality of overlapping oligonucleotides is in the range of about 0.8 to about 4.0 μM.

6. The method of claim 3, wherein the selected DNA polymerase has an error frequency of about 0.01% or less.

7. The method of claim 1, further comprising the step of diluting the template DNA after the step of assembling the plurality of overlapping oligonucleotides and prior to the step of amplifying the template DNA.

8. The method of claim 1, further comprising, as a first step, the step of optimizing the plurality of overlapping oligonucleotides.

9. The method of claim 8, wherein the step of optimizing the plurality of overlapping oligonucleotides is accomplished by defining a host in which the synthesized gene of interest will be expressed, and optimizing codons of the plurality of overlapping oligonucleotides with respect to the defined host.

10. The method of claim 8, wherein the step of optimizing the plurality of overlapping oligonucleotides comprises altering the nucleotide sequence of at least one of the plurality of overlapping oligonucleotides such that the nucleotide sequence of the template DNA differs in at least one codon from the nucleotide sequence of the gene of interest.

11. The method of claim 10, wherein the at least one codon of the template DNA is a codon with optimal frequency of usage in the defined host, and wherein the template DNA encodes a protein having an amino acid sequence identical to the amino acid sequence of the protein encoded by the gene of interest.

12. The method of claim 10, wherein the at least one codon of the template DNA introduces a mutation into the protein encoded by the gene of interest.

13. The method of claim 1, wherein the gene of interest is about 300 to about 1700 base pairs in length.

14. The method of claim 1, wherein the gene of interest is selected from the group consisting of FAAH, hDAOA, pDAO, hCatSper3, GPR55, TREM2, IGF1, USAG1, IGFBP4, and mTPH2.

15. The method of claim 3, wherein the gene of interest is selected from the group consisting of FAAH, hDAOA, pDAO, hCatSper3, GPR55, TREM2, IGF1, USAG1, IGFBP4, and mTPH2.

16. The method of claim 15, wherein the gene of interest is selected from the group consisting of FAAH and GPR55.

17. A nucleic acid molecule comprising the gene of interest synthesized according to the method of claim 1.

18. A method of producing a polypeptide, comprising the following steps:
(a) culturing a host cell comprising the gene of interest synthesized according to the method of claim 1 under conditions such that the polypeptide is expressed; and
(b) purifying the expressed polypeptide from the host cell.

19. A polypeptide produced by the method of claim 18.
